(19) United States
(12) Patent Application Publication
(10) Pub. No.: US 2018/0317749 A1
(43) Pub. Date: Nov. 8, 2018

(54) ENDOSCOPE

(71) Applicant: OLYMPUS CORPORATION, Tokyo (JP)

(72) Inventors: Masami Shimizu, Tokyo (JP); Shigeyasu Kishioka, Tokyo (JP); Yoki Onishi, Tokyo (JP)

(73) Assignee: OLYMPUS CORPORATION, Tokyo (JP)

(21) Appl. No.: 16/038,713

(22) Filed: Jul. 18, 2018

Related U.S. Application Data
(63) Continuation of application No. PCT/JP2016/078081, filed on Sep. 23, 2016.

(30) Foreign Application Priority Data
Jan. 20, 2016 (JP) 2016-008630

(51) Int. Cl. A61B 1/00; A61B 1/005; A61B 1/04

(52) U.S. Cl. CPC A61B 1/00052; A61B 1/121; A61B 1/042; A61B 1/0052

(57) ABSTRACT

A video display apparatus includes an apparatus main body, a lid body, a hinge shaft configured to axially support the lid body with respect to the apparatus main body, main body-side shaft holding sections, the hinge shaft being inserted through the main body-side shaft holding sections, lid-side shaft holding sections formed in the lid body, the hinge shaft being inserted through the lid-side shaft holding sections, a first seal member configured to prevent infiltration of liquid from a gap between the lid-side shaft holding sections and the main body-side shaft holding sections into the hinge shaft, a hinge lid disposed in a stepped hole, into which the hinge shaft is inserted, provided in one of the lid-side shaft holding sections, and a second seal member provided in the stepped hole and configured to prevent infiltration of the liquid into the hinge shaft.

### CROSS REFERENCE TO RELATED APPLICATION

This application is a continuation application of PCT/JP2016/078081 filed on Sep. 23, 2016 and claims benefit of Japanese Application No. 2016-008630 filed in Japan on Jan. 20, 2016, the entire contents of which are incorporated herein by this reference.

### BACKGROUND OF THE INVENTION

### 1. Field of the Invention

The present invention relates to an endoscope integrally attached with a video display apparatus.

### 2. Description of the Related Art

In recent years, endoscopes have been used in a medical field, an industrial field, and the like. Among endoscopes, there are a videoscope in which an image pickup apparatus is incorporated in a distal end portion of an insertion section and a fiberscope in which an image guide obtained by bundling optical fibers is inserted through an insertion section.

Japanese Patent Application Laid-Open Publication No. 2005-192738 discloses an endoscope apparatus integrated with a small video display apparatus and suitable for portable use. The video display apparatus is attached to an operation section of an endoscope. A display device such as an LCD, an image recording apparatus, an image pickup device control circuit, and a display device control circuit are incorporated in the video display apparatus. A battery configured to supply electric power to a light source apparatus, an image pickup device, the display device, the image recording apparatus, and various control circuits is detachably incorporated inside a housing of the video display apparatus.

Loading sections for loading the battery and a recording medium are provided inside the housing of the video display apparatus. Loading ports for enabling the battery and the recording medium to be loaded are closed by a lid body.

The lid body is openably and closably coupled to the housing via a hinge section. More specifically, the lid body is structured to be turnably supported by being coupled to a seat plate fixed to the housing via a pin. The lid body is prevented from unexpectedly opening by a lock mechanism. The lock mechanism includes an engaging section including a recessed section engageable with a claw section provided at the other side edge of the lid body coupled to the hinge section and a lever configured to draw the engaging section engaged with the claw section to the housing side and fix the engaging section in a home position.

When an endoscope apparatus suitable for portable use is for, for example, medical use, the endoscope apparatus can be, for example, used in a treatment room or a sickroom in addition to an operating room. An endoscope used for medical use is sufficiently washed and disinfected to be cleaned after the use.

### SUMMARY OF THE INVENTION

An endoscope according to an aspect of the present invention includes a video display apparatus attached to an operation section provided on a proximal end side of an endoscope insertion section. The video display apparatus includes: an apparatus main body including a housing section; a lid body capable of closing an opening of the housing section; a hinge shaft configured to axially support the lid body turnably with respect to the apparatus main body; main body-side shaft holding sections formed further on an outer side than the opening of the apparatus main body and including holes through which the hinge shaft is inserted and disposed; lid-side shaft holding sections formed in the lid body, including holes through which the hinge shaft is inserted and disposed, and provided adjacent to the main body-side shaft holding sections; a first seal member interposed on an outer circumference side of the hinge shaft and between the lid-side shaft holding sections and the main body-side shaft holding sections and configured to prevent infiltration of liquid from a gap between the lid-side shaft holding sections and the main body-side shaft holding sections into the hinge shaft; a hinge lid disposed in a hinge shaft insertion port section, into which the hinge shaft is inserted, provided in one of the lid-side shaft holding sections or the main body-side shaft holding sections; and a second seal member provided in the hinge shaft insertion port section and configured to prevent infiltration of the liquid from a gap between the hinge shaft insertion port section and the hinge lid into the hinge shaft.

### BRIEF DESCRIPTION OF THE DRAWINGS

FIG. 1 is a two-view diagram, that is, a front view and a left side view with respect to the front view for explaining an endoscope including a video display apparatus;

FIG. 2 is an enlarged view for explaining the video display apparatus described in the left side view of FIG. 1;

FIG. 3 is a diagram for explaining an apparatus main body in which a lid body is set in an opened state

FIG. 4A is a diagram for explaining a hinge section configured to turnably couple the lid body to the apparatus main body, the diagram including an arrow Y4A-Y4A line sectional view of FIG. 2;

FIG. 4B is a diagram for explaining a relation between a stepped hole provided in a first lid-side shaft holding section and a hinge lid and a second O-shaped ring;

FIG. 5A is a diagram for explaining an assembly procedure and is a diagram showing a main body-side shaft holding section in which a first O-shaped ring is disposed and the hinge lid in which the second O-shaped ring is disposed;

FIG. 5B is a diagram for explaining the assembly procedure and is a diagram showing a state in which the first lid-side shaft holding section and a second lid-side shaft holding section of the lid body are held and disposed with respect to the main body-side shaft holding section;

FIG. 5C is a diagram for explaining the assembly procedure and is a diagram for explaining a hinge shaft, a shaft hole through which the hinge shaft is inserted and disposed, a shaft through-hole, and a stop hole;

FIG. 5D is a diagram for explaining the assembly procedure and is a diagram for explaining a relation between the hinge shaft disposed in the shaft hole, the shaft through-hole, and the stop hole and the hinge lid in which the second O-shaped ring is disposed;

FIG. 5E is a diagram showing an assembly completed state and is a diagram for explaining the hinge shaft disposed in a watertight space;

FIG. 6 is a diagram of the video display apparatus viewed from an arrow Y6 direction in FIG. 2;

FIG. 7A is a diagram showing a modification of the hinge shaft;

FIG. 7B is a diagram for explaining a modification of a second lid-side shaft holding section;

FIG. 7C is a diagram for explaining a modification of the hinge section and a configuration example including one lid-side shaft holding section and a pair of main body-side shaft holding sections;

FIG. 8 is a diagram for explaining a lid body including an opening and closing apparatus and the apparatus main body;

FIG. 9 is a diagram for explaining a closed state in which the lid body closes a housing opening of the apparatus main body;

FIG. 10 is a Y10-Y10 line sectional view of FIG. 9 and is a diagram for explaining a cam surface provided on an apparatus main body rear side surface; and

FIG. 11 is a diagram for explaining a relation between an inclined surface and a flat surface of the cam surface.

### DETAILED DESCRIPTION OF THE PREFERRED EMBODIMENT

An embodiment of the present invention is explained below with reference to the drawings.

Note that, in respective figures used in the following explanation, scales are sometimes differentiated for each of components to show the respective components in sizes recognizable on the drawings. The present invention is not limited only to numbers of the components, shapes of the components, ratios of sizes of the components, and relative positional relations among the respective components described in the figures.

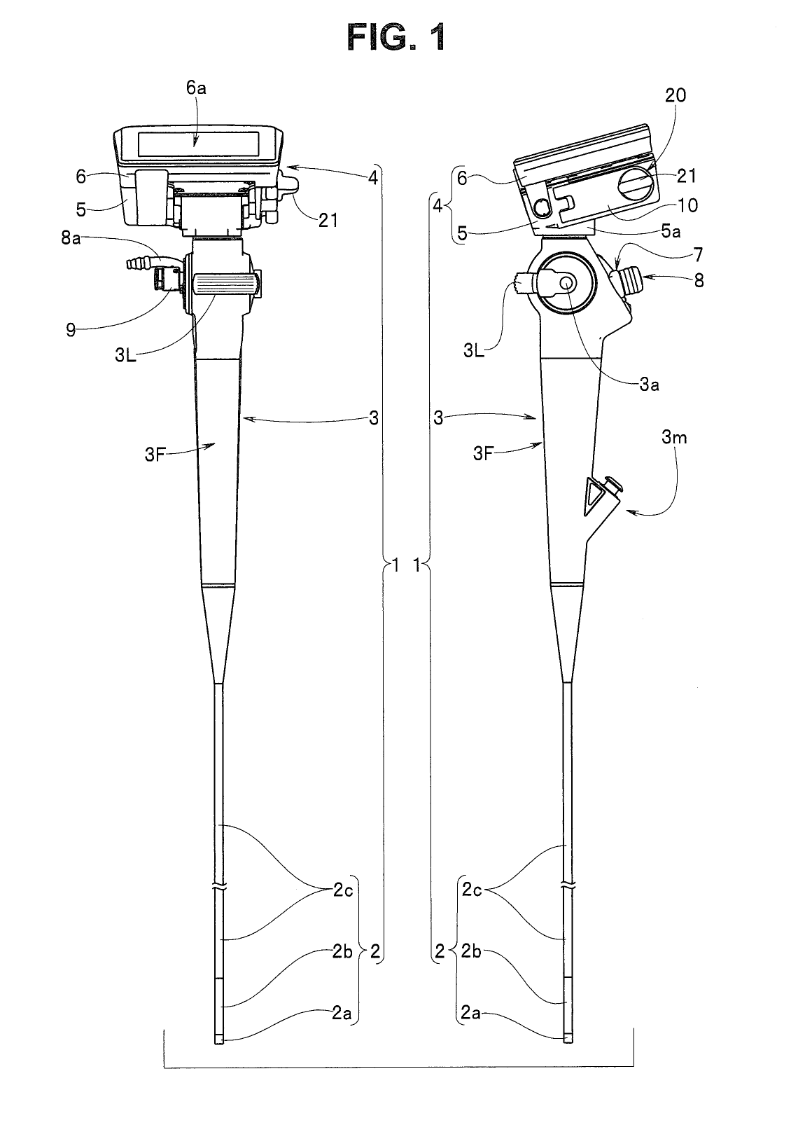

An endoscope 1 shown in FIG. 1 is a so-called portable endoscope and mainly includes an insertion section 2, an operation section 3, and a video display apparatus 4.

In the insertion section 2, a distal end portion 2a, a bending section 2b, and a flexible tube section 2c are concatenated in order from a distal end side. The flexible tube section 2c is a tube body having predetermined flexibility. The bending section 2b is configured to bend in, for example, an up-down direction.

Reference sign 3L denotes a bending operation lever. The bending operation lever 3L can be operated by a finger, for example, a thumb of a hand of a user who grips the operation section 3. The bending operation lever 3L is capable of turning around an axis 3a. A bending wire (not shown in the figure) is pulled and loosened according to turning operation of the bending operation lever 3L, whereby the bending section 2b bends in an upward direction or a downward direction.

Note that reference numeral 3m denotes a treatment instrument insert-through opening and an introduction opening for inserting an endoscope treatment instrument such as forceps. Reference numeral 3F denotes an operation section front surface. The operation section front surface 3F is a surface opposed to a front of the user when the user operates the bending operation lever 3L.

The video display apparatus 4 includes an apparatus main body 5 and a display section 6. A display screen 6a is provided on a front side surface, which is an upper surface in the figure, of the display section 6. The display section 6 is axially supported turnably with respect to the apparatus main body 5. Therefore, a direction of the display screen 6a of the display section 6 is changeable with respect to a surgeon.

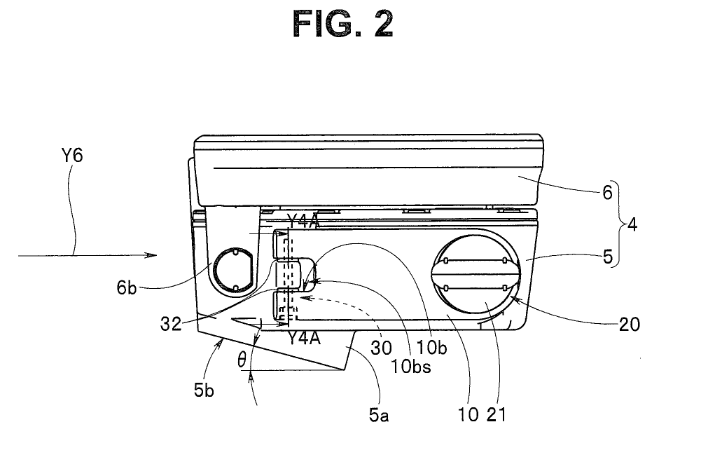

The apparatus main body 5 includes a main body attaching section 5a. One surface of the main body attaching section 5a is attached and fixed to a proximal end face of the operation section 3. A lid body 10 configured to close a housing space 5S, which is a housing section, is turnably attached to the apparatus main body 5 via a hinge section (see reference numeral 30 in FIG. 2). Reference numeral 21 denotes a knob of an opening and closing apparatus 20. The knob 21 is provided on a right side viewed from the operation section front surface 3F such that, when the user grips the operation section 3 with a left hand, the user can easily operate the knob 21 with a right hand in a state in which the operation section front surface 3F is opposed to a front of a body.

Note that reference numeral 7 denotes a suction pipe sleeve. A suction valve 8 for sucking body fluid or the like is attached to the suction pipe sleeve 7. The suction valve 8 includes a connecting section 8a. A suction tube extended from a not-shown suction apparatus is connected to the connecting section 8a. Reference numeral 9 denotes a ventilation pipe sleeve. The ventilation pipe sleeve 9 is used in a water leak inspection. An air supply tube extended from a not-shown air supply apparatus is connected to the ventilation pipe sleeve 9.

The video display apparatus 4 is explained with reference to FIG. 1 to FIG. 4B.

As shown in FIG. 1, the video display apparatus 4 is attached and fixed to the proximal end face of the operation section 3. In a state in which the user grips the operation section 3 and suspends the insertion section 2 in a vertical direction, the display screen 6a of the display section 6 set on an upper surface of the apparatus main body 5 is disposed to tilt at a predetermined angle with respect to the horizontal to be opposed to the front of the user.

That is, as shown in FIG. 2, a placing surface 5b, which is the one surface of the main body attaching section 5a, is formed as an inclined surface having a predetermined angle θ.

The lid body 10 is in a closed state and can be opened by rotating the knob 21 90 degrees clockwise.

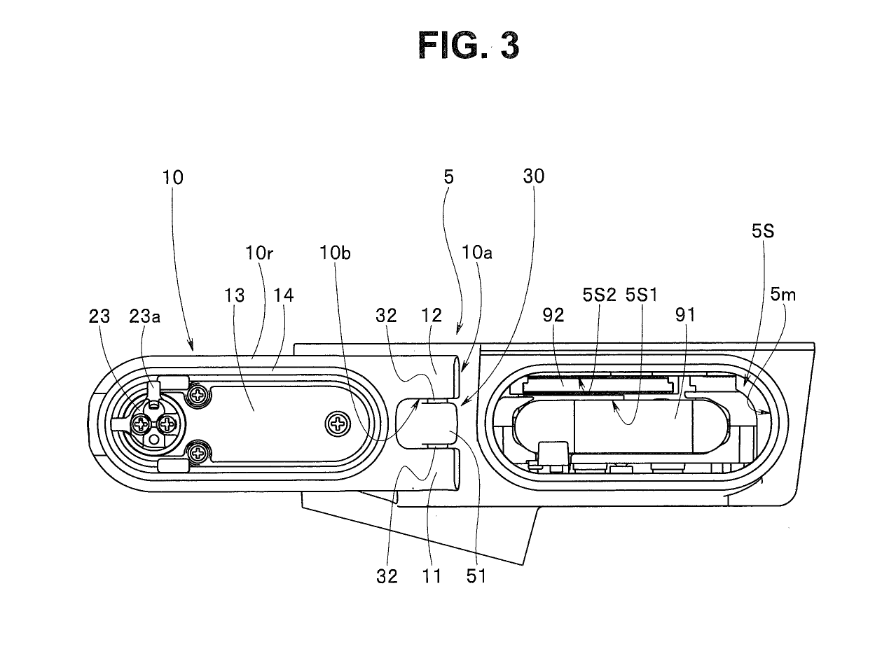

The housing space 5S is exposed as shown in FIG. 3 by setting the lid body 10 in an opened state.

The housing space 5S includes a battery housing section 5S1 and a medium housing section 5S2. A battery 91 is loaded in the battery housing section 5S1. A recording medium 92 is loaded in the medium housing section 5S2.

The battery 91 supplies electric power to a display device, an image recording apparatus, a control circuit, and a light source apparatus. Reference sign 5m denotes a housing opening.

A rear plate 13 is integrally provided on a rear surface 10r side of the lid body 10. A watertight protection member 14, which is an elastic member and disposed along an outer side of the housing opening 5m, is disposed on the rear plate 13. The watertight protection member 14 is disposed in close contact with the outer side of the housing opening 5m of the apparatus main body 5 when the lid body 10 is set in the closed state. Because the watertight protection member 14 is disposed in close contact, liquid such as water is prevented from infiltrating into the housing space 5S.

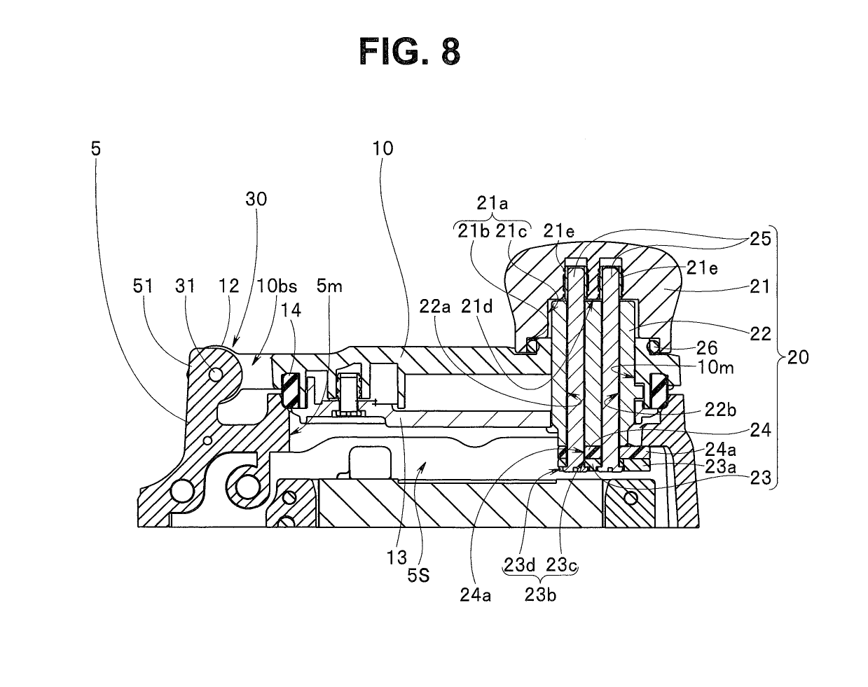

A shaft (see reference numeral 22 in FIG. 8) integrally provided in the knob 21, a lock plate 23, and the like are disposed on the rear plate 13. Reference sign 23a denotes a locking claw. The locking claw 23a projects by a predetermined amount from an outer circumferential surface of the lock plate 23.

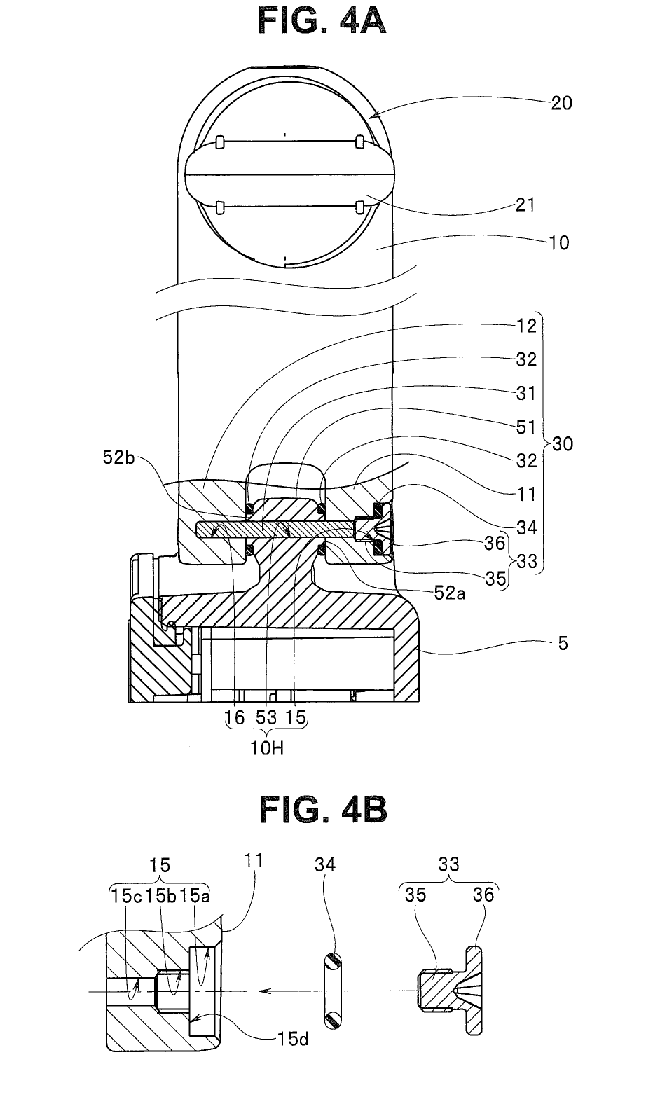

As shown in FIG. 3 and FIG. 4A, the hinge section 30 includes a hinge shaft 31, a main body-side shaft holding section 51, a pair of lid-side shaft holding sections 11 and 12, a pair of first seal members 32, a hinge lid 33, and one second seal member 34.

The hinge shaft 31 is a shaft member, a sectional outer shape of which is a circle. The hinge shaft 31 is set to a predetermined length. The first seal members 32 are elastic members having a predetermined elastic force and are, for example, O-shaped rings (hereinafter described as first O-shaped rings 32). The hinge lid 33 is a so-called screw-like member including a shaft section 35 and a flange section 36. The shaft section 35 is set to a predetermined length and formed as a male screw section. The second seal member 34 is also an elastic member having a predetermined elastic force and is, for example, an O-shaped ring (hereinafter described as second O-shaped ring 34).

The main body-side shaft holding section 51 is provided in the apparatus main body 5. The main body-side shaft holding section 51 is a protrusion section provided in a predetermined position further on an outer side than the housing opening 5m of the housing space 5S. Projecting sections 52a and 52b are provided in the main body-side shaft holding section 51. The first O-shaped rings 32 are respectively disposed in the projecting sections 52a and 52b.

The thickness and the elastic force of the first O-shaped rings 32 disposed in the projecting sections 52a and 52b are set such that, in a squashed state, the first O-shaped rings 32 further project than end faces of the projecting sections 52a and 52b by a predetermined dimension.

On the other hand, the first lid-side shaft holding section 11 and the second lid-side shaft holding section 12 are shaped by forming a recessed section 10b in an intermediate portion of one short side 10a of the lid body 10. The main body-side shaft holding section 51 is disposed in the recessed section 10b.

That is, the main body-side shaft holding section 51 is disposed between the first lid-side shaft holding section 11 and the second lid-side shaft holding section 12. In this disposition state, the first O-shaped rings 32 are disposed between the main body-side shaft holding section 51 and the first lid-side shaft holding section 11 and between the main body-side shaft holding section 51 and the second lid-side shaft holding section 12.

Holes for inserting through and disposing the hinge shaft 31 are respectively formed in the main body-side shaft holding section 51, the first lid-side shaft holding section 11, and the second lid-side shaft holding section 12.

The hole formed in the main body-side shaft holding section 51 is a shaft through-hole 53 and a halfway portion of the hinge shaft 31 is disposed therein by predetermined fitting. Openings of the shaft through-hole 53 are respectively formed on an end face of the first projecting section 52a and an end face of the second projecting section 52b.

The hole formed in the second lid-side shaft holding section 12 is a stop hole 16 and an end portion of the hinge shaft 31 is disposed therein by predetermined fitting. Depth of the stop hole 16 is set to a predetermined dimension.

The hole formed in the first lid-side shaft holding section 11 is a stepped hole 15 and is a hinge shaft insertion port section. As shown in FIG. 4B, the stepped hole 15 includes a first hole 15a, a second hole 15b, and a shaft hole 15c. The first hole 15a has a largest diameter. The shaft hole 15c has a smallest diameter. The second hole 15b has an intermediate inner diameter between the diameters of the first hole 15a and the shaft hole 15c.

An end portion of the hinge shaft 31 is disposed in the shaft hole 15c. The second O-shaped ring 34 and the hinge lid 33 are disposed in the first hole 15a. The shaft section 35 of the hinge lid 33 is disposed in the second hole 15b. A female screw section configured to screw with the male screw section of the shaft section 35 is formed on an inner circumferential surface of the second hole 15b.

Note that the second O-shaped ring 34 is disposed on a bottom surface 15d side of the first hole 15a.

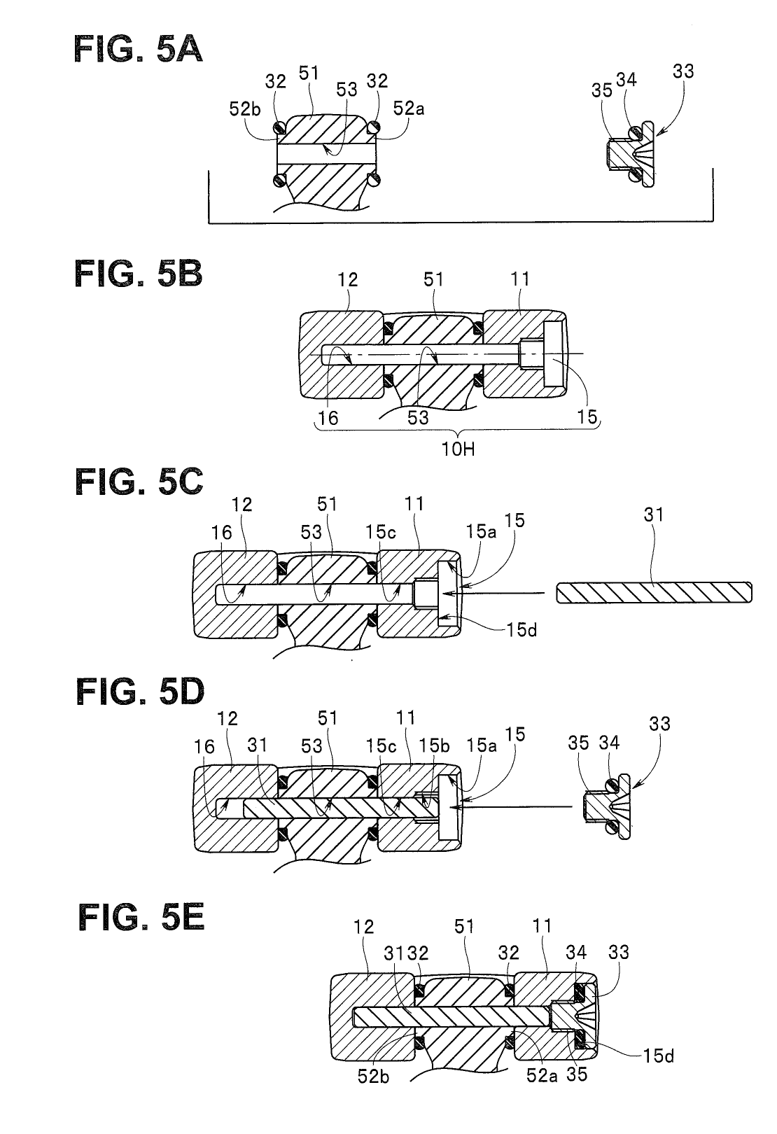

An example of assembly of the lid body 10 to the apparatus main body 5 is explained with reference to FIG. 5A to FIG. 5E.

As shown in FIG. 5A, an operator disposes the first O-shaped rings 32 in the first projecting section 52a and the second projecting section 52b of the main body-side shaft holding section 51. The operator disposes the second O-shaped ring 34 on a root side of the shaft section 35 of the hinge lid 33.

Subsequently, the operator disposes the lid-side shaft holding sections 11 and 12 of the lid body 10 in the main body-side shaft holding section 51 while resisting an elastic force of the first O-shaped rings 32. As shown in FIG. 5B, the operator disposes the lid-side shaft holding sections 11 and 12 to hold the main body-side shaft holding section 51 to form a shaft insert-through hole 10H in a state in which a center axis of the stop hole 16, a center axis of the shaft through-hole 53, and a center axis of the stepped hole 15 substantially coincide with one another.

In the holding and disposition state described above, the two first O-shaped rings 32 are respectively squashed and elastically deformed. That is, one first O-shaped ring 32 adheres to a side surface of the main body-side shaft holding section 51 and a side surface of the first lid-side shaft holding section 11. The other first O-shaped ring 32 adheres to the side surface of the main body-side shaft holding section 51 and a side surface of the second lid-side shaft holding section 12.

As a result, a gap between the main body-side shaft holding section 51 and the first lid-side shaft holding section 11 is closed by the one first O-shaped ring 32. The shaft insert-through hole 10H is maintained in a watertight state for preventing liquid such as water from infiltrating into the shaft insert-through hole 10H from the gap. On the other hand, a gap between the main body-side shaft holding section 51 and the second lid-side shaft holding section 12 is closed by the other first O-shaped ring 32. The shaft insert-through hole 10H is maintained in a watertight state for preventing liquid such as water from infiltrating into the shaft insert-through hole 10H from the gap.

Subsequently, as shown in FIG. 5C, the operator inserts the hinge shaft 31 through the shaft hole 15c, the shaft through-hole 53, and the stop hole 16, which configure the shaft insert-through hole 10H, from the first hole 15a side of the stepped hole 15. Then, the hinge shaft 31 is disposed in the shaft insert-through hole 10H as shown in FIG. 5D.

Subsequently, as shown in FIG. 5D, the operator disposes the shaft section 35 of the hinge lid 33, in which the second O-shaped ring 34 is provided, in the shaft hole 15c from the first hole 15a side of the stepped hole 15. Thereafter, the operator screws the shaft section 35 into the shaft hole 15c while rotating the hinge lid 33 in a predetermined direction with, for example, a flat head screwdriver.

Then, a distal end face of the hinge shaft 31 is moved toward a bottom surface of the stop hole 16. The flange section 36 of the hinge lid 33 is gradually moved toward the bottom surface 15d. The flange section 36 comes into contact with the second O-shaped ring 34 and gradually squashes the O-shaped ring 34.

Thereafter, the operator continues to rotate the hinge lid 33 in the same direction, whereby the shaft section 35 is fixed in the shaft hole 15c in a predetermined screwed state as shown in FIG. 5E.

As a result, the distal end face of the hinge shaft 31 is disposed in the stop hole 16 in a predetermined state. The second O-shaped ring 34 is elastically deformed by the flange section 36 and adheres to an outer circumferential surface of the shaft section 35 and the bottom surface 15d of the first hole 15a. The shaft insert-through hole 10H is maintained in a watertight state for preventing liquid from infiltrating into the shaft insert-through hole 10H from the first hole 15a side.

That is, an inside of the shaft insert-through hole 10H, which is a sliding portion in which the hinge shaft 31 is disposed, is formed as a sealed space maintained watertight by the O-shaped rings 32 and 33.

Therefore, the hinge shaft 31 of the hinge section 30 configured to axially support the lid body 10 turnably with respect to the apparatus main body 5 is surely prevented from coming into contact with liquid such as water. Therefore, it is unnecessary to wash the hinge shaft 31 of the hinge section 30.

Note that, as shown in FIG. 2 and FIG. 4A, an outer circumferential surface of the first O-shaped ring 32 is exposed to an outside. Therefore, in washing work, washing can be performed by bringing a bristle tip of a washing brush (not shown in the figures) into contact with a surface of the first O-shaped ring 32.

In a state in which the main body-side shaft holding section 51 is disposed in the recessed section 10b as shown in FIG. 2, a space 10bs is formed in the recessed section 10b. The space 10bs has size for enabling the washing brush to pass. Therefore, in the washing work, the washing brush can be disposed in the space 10bs to perform washing of an inner surface of the recessed section 10b, an outer surface of the main body-side shaft holding section 51, and the like.

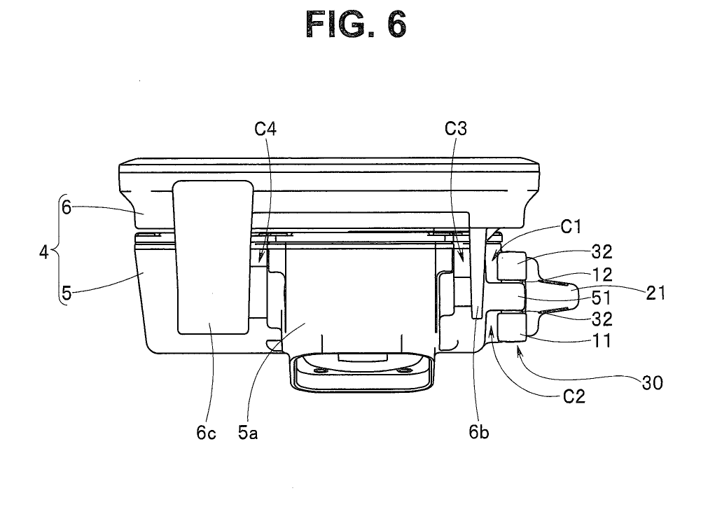

As shown in FIG. 6, a first gap C1 and a second gap C2 between the hinge section 30 and a first monitor supporting protrusion 6b have size for enabling the washing brush to pass. In addition, a third gap C3 between the main body attaching section 5a and the first monitor supporting protrusion 6b also has size for enabling the washing brush to pass. A fourth gap C4 between the main body attaching section 5a and a second monitor supporting protrusion 6c also has size for enabling the washing brush to pass. Therefore, in the washing work, the washing brush can be disposed in the gaps C1, C2, C3, and C4 to perform washing.

In this way, in the endoscope 1 in which the video display apparatus 4 is provided in the operation section 3, the hinge shaft 31 configuring the hinge section 30 is disposed in the shaft insert-through hole 10H, which is the sealed space maintained watertight. The outer circumferential surface of the first O-shaped ring 32 for forming the shaft insert-through hole 10H as the sealed space is exposed to the outside. In addition, the space 10bs for enabling the washing brush to pass and the gaps C1, C2, C3, and C4 are provided to separate components in advance.

As a result, it is possible to easily perform washing of the gap between the main body-side shaft holding section 51 and the first lid-side shaft holding section 11 and the gap between the main body-side shaft holding section 51 and the second lid-side shaft holding section 12. That is, it is possible to easily perform washing and disinfection of the hinge section 30 by washing and disinfecting an outer surface around the hinge shaft 31 without washing and disinfecting the hinge shaft 31.

Therefore, in the endoscope 1, it is possible to easily perform washing and disinfection of the video display apparatus 4 in addition to washing and disinfection of the insertion section 2 and the operation section 3.

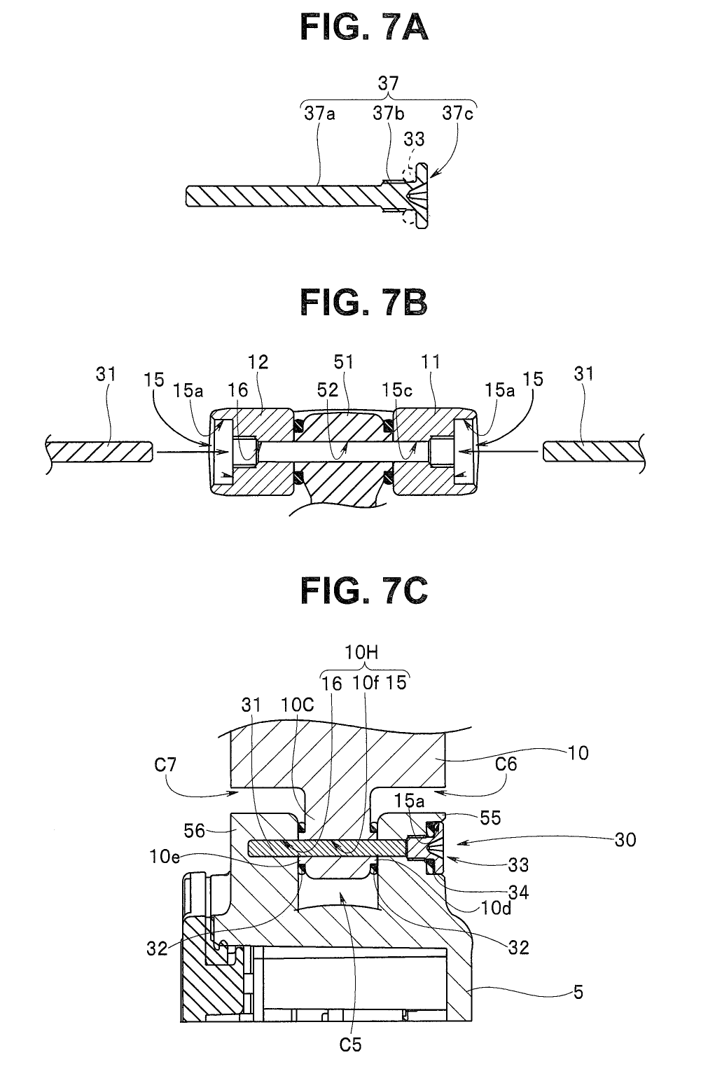

Note that, in the embodiment explained above, the hinge shaft 31 and the hinge lid 33 are formed as separate bodies. However, a hinge lid with hinge shaft 37 may be configured as shown in FIG. 7A.

The hinge lid with hinge shaft 37 includes a hinge shaft section 37a corresponding to the hinge shaft 31, a screw section 37b corresponding to the shaft section 35 including the male screw section, and a flange section 37c similar to the flange section 36.

With this configuration, work for disposing the hinge shaft and work for disposing the hinge lid can be performed at a time.

As shown in FIG. 7B, the stepped hole 15 may be provided in the second lid-side shaft holding section 12 in addition to the first lid-side shaft holding section 11.

Consequently, when the hinge shaft 31 or the hinge shaft section 37a of the hinge lid with hinge shaft 37 is disposed in the shaft insert-through hole 10H, it is possible to perform insert-through disposition work from the second lid-side shaft holding section 12 side in addition to the first lid-side shaft holding section 11 side. When the hinge shaft 31 is detached, it is also possible to easily detach the hinge shaft by inserting a pin or the like from one hole and pushing the hinge shaft.

As a result, it is possible to achieve improvement of assemblability. Note that the stepped hole 15 of one lid-side shaft holding section in an open state is maintained in a watertight state by the second O-shaped ring 34 by screwing and disposing the hinge lid 33.

In the embodiment explained above, a pair of lid-side shaft holding sections 11 and 12 is provided in the lid body 10 and the main body-side shaft holding section 51, which is one protrusion section, is provided in the main body 5. However, as shown in FIG. 7C, main body-side shaft holding sections 55 and 56, which are two protrusion sections, may be provided in the main body 5 and one lid-side shaft holding section 10C may be provided in the lid body 10 to configure the hinge section 30.

In the lid-side shaft holding section 10C, projecting sections 10d and 10e, in which the O-shaped rings 32 corresponding to the projecting sections 52a and 52b are respectively disposed, are provided. A shaft through-hole 10f corresponding to the shaft through-hole 53 is provided in the lid-side shaft holding section 10C.

On the other hand, the stepped hole 15 is provided in the first main body-side shaft holding section 55. The stop hole 16 is provided in the second main body-side shaft holding section 56.

With this configuration, the operator disposes the lid-side shaft holding section 10C between the first main body-side shaft holding section 55 and the second main body-side shaft holding section 56 while resisting the elastic force of the first O-shaped rings 32.

As a result, the shaft insert-through hole 10H, through which the hinge shaft 31 is inserted and disposed, is configured by the stepped hole 15, the shaft through-hole 10f, and the stop hole 16.

Note that reference signs C5, C6, and C7 denote a fifth gap, a sixth gap, and a seventh gap having size for enabling the washing brush to pass.

With this configuration, it is possible to realize the endoscope 1 excellent in washability including the same hinge section 30 as the hinge section 30 in the embodiment explained above.

Incidentally, in Japanese Patent Application Laid-Open Publication No. 2005-192738, a lock mechanism is provided to prevent a lid body from unexpectedly opening during use, during washing and disinfection, or the like. In the lock mechanism, a lever and an engaging section are in a substantial adhesion state to a lower surface of a housing. Therefore, when an endoscope is washed and disinfected, it is difficult to wash and disinfect a gap between the lever and the housing and a gap between the engaging section and the housing.

In the lid body 10 explained above, a lock mechanism for preventing, without deteriorating washability, the lid body from opening under an unexpected situation during use, during washing and disinfection, or the like is provided.

As shown in FIG. 8, the opening and closing apparatus 20 includes the knob 21, the shaft 22, the lock plate 23, a resin plate 24, and a pair of fixing screws 25.

The knob 21 includes a stepped hole 21a, a lid hole 21b having a large diameter, and a shaft hole 21c having a diameter smaller than the hole 21b. Two female screw sections 21e, in which male screw sections provided on one end portion sides of fixing screws 25 are screwed, are provided in predetermined positions of a bottom surface 21d of the shaft hole 21c.

One end portion of the shaft 22 is disposed in the shaft hole 21c of the knob 21. A halfway portion of the shaft 22 is disposed in an opening and closing apparatus hole 10m provided in the lid body 10. The other end portion of the shaft 22 projects from a rear surface side of the lid body 10.

The other end portion of the shaft 22 is located in the housing space 5S when the lid body 10 is in the closed state.

In the shaft 22, two through-holes 22a and 22b, in which shaft sections of the fixing screws 25 are disposed, are formed along a longitudinal axis direction.

The lock plate 23 is a plate made of metal including the locking projecting section 23a explained above. The lock plate 23 is substantially circular. The locking projecting section 23a has a predetermined shape and projects outward from an outer circumferential surface of the lock plate 23.

Two counterbores 23b corresponding to the through-holes 22a and 22b of the shaft 22 are formed on one surface of the lock plate 23. The counterbores 23b include holes 23c and through-holes 23d. Flanges 25f provided at proximal end portions of the fixing screws 25 are disposed in the holes 23c. Shaft sections of the fixing screws 25 are disposed in the through-holes 23d.

The resin plate 24 is a member for reducing friction. The resin plate 24 has substantially the same circular shape as the lock plate 23. A projecting section 24a having substantially the same shape as the locking projecting section 23a is provided in the resin plate 24. Two through-holes 24b, in which the shaft sections of the fixing screws 25 are disposed, are formed in the resin plate 24.

The opening and closing apparatus 20 is integrally attached to the lid body 10.

More specifically, the halfway portion of the shaft 22 is disposed in the opening and closing apparatus hole 10m. In this state, one end portion of the shaft 22 is disposed in the shaft hole 21c of the knob 21 in a predetermined state. On the other hand, the resin plate 24 and the lock plate 23 are disposed on the other end face of the shaft 22 in a predetermined state. Thereafter, the fixing screws 25 are inserted through from the counterbores 23b side of the lock plate 23 to screw and fix male screw sections of the screws 25 in the female screw sections 21e. As a result, the opening and closing apparatus 20 is provided integrally in the lid body 10.

Reference numeral 26 denotes an O-shaped ring. The O-shaped ring 26 maintains a gap between the lid body 10 and the knob 21 watertight.

With this configuration, the knob 21, the shaft 22, the resin plate 24, and the lock plate 23 are integrated. That is, by rotating the knob 21, the projecting section 24a of the resin plate 24 and the locking projecting section 23a of the lock plate 23 integrally turn.

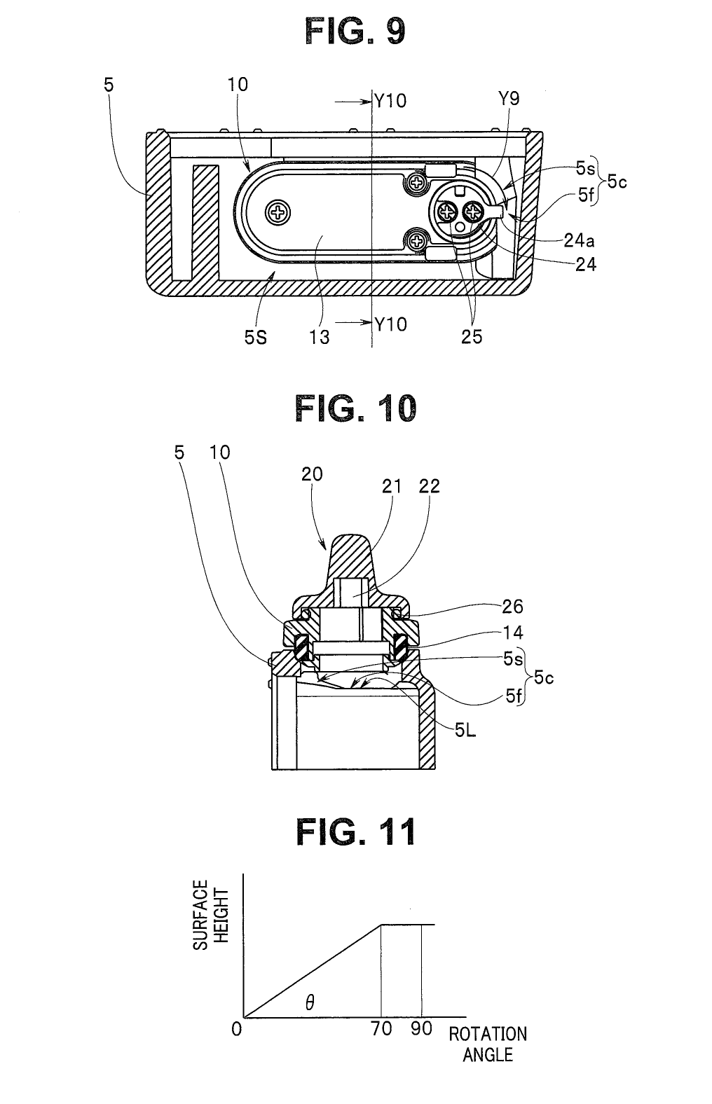

As shown in FIG. 9, the projecting section 24a of the resin plate 24 and the projecting section 23a of the lock plate 23 of the opening and closing apparatus 20 are rotated 90 degrees as indicated by an arrow Y9 to be disposed in a closed position.

The projecting section 24a of the resin plate 24 is disposed slidably with respect to a cam surface 5c provided on an apparatus main body rear side surface around the housing opening 5m shown in FIG. 10. The cam surface 5c is a lock mechanism and includes an inclined surface 5s and a flat surface 5f on which the projecting section 24a of the resin plate 24 slides. When the knob 21 is turned, friction between the projecting section 24a of the resin plate 24 and the cam surface 5c is reduced. The knob 21 can be smoothly turned.

As shown in FIG. 11, the inclined surface 5s is provided between 0 degree and 70 degrees of a rotation angle of the knob 21. On the other hand, the flat surface 5f is provided between 70 degrees and 90 degrees of the rotation angle of the knob 21. The flat surface 5f is a watertightness maintaining surface for maintaining a watertight state.

Note that the inclined surface 5s is formed such that a surface height continuously gradually becomes higher than a reference plane. The flat surface 5f maintains a largest surface height of the inclined surface 5s. The inclined surface 5s and the flat surface 5f are formed to continue as a smooth surface.

That is, in a state in which the projecting section 24a of the resin plate 24 is disposed on the flat surface 5f, the knob 21 of the opening and closing apparatus 20 closely attaches the watertight protection member 14 to an outer side of the housing opening 5m of the apparatus main body 5 to set the lid body 10 in the closed state with respect to the apparatus main body 5. The housing space 5S is maintained in a watertight state in which liquid such as water is prevented from infiltrating into the housing space 5S.

When the user closes the lid body 10 in an open state, the lid body 10 is disposed to close the housing opening 5m. At this time, the watertight protection member 14 is disposed on the outer side of the housing opening 5m of the apparatus main body 5. The user rotates the knob 21, whereby an adhesion degree of the watertight protection member 14 and the outer side of the housing opening 5m gradually increases.

The user rotates the knob 21 to a position of 90 degrees, which is a position where the user can surely visually recognize that the lid body 10 is in the closed state. Then, the lid body 10 comes into the closed state explained above and is maintained in the watertight state explained above.

In the watertight state, even if the user touches the lid body 10 and moves a position of the lid body 10 from the position of 90 degrees to a position around 70 degrees, the closed state is maintained and the watertight state is maintained while the projecting section 24a is located on the flat surface 5f.

That is, it is possible to surely prevent, without deteriorating the washability of the video display apparatus 4 including the lid body 10, the lid body 10 from unexpectedly opening during use, during washing and disinfection, or the like.

Note that, in the embodiment explained above, the rotation angle of the watertightness maintaining surface is set in a range of 20 degrees from 70 degrees to 90 degrees. However, the watertightness maintaining surface may be changed as appropriate, for example, from 70 degrees to 100 degrees or from 65 degrees to 105 degrees.

An inclination angle of the inclined surface 5s is not limited to the angle shown in FIG. 11 and is set as appropriate.

Note that the present invention is not limited only to the embodiment explained above and can be variously modified and carried out in a range not departing from the spirit of the invention.

According to the present invention, it is possible to realize an endoscope in which washing work of a hinge section of a lid body provided in a video display apparatus can be easily and surely performed.

What is claimed is:

1. An endoscope comprising a video display apparatus attached to an operation section provided on a proximal end side of an endoscope insertion section, wherein
the video display apparatus includes:
an apparatus main body including a housing section;
a lid body capable of closing an opening of the housing section;
a hinge shaft configured to axially support the lid body turnably with respect to the apparatus main body;
main body-side shaft holding sections formed further on an outer side than the opening of the apparatus main body and including holes through which the hinge shaft is inserted and disposed;
lid-side shaft holding sections formed in the lid body, including holes through which the hinge shaft is inserted and disposed, and provided adjacent to the main body-side shaft holding sections;
a first seal member interposed on an outer circumference side of the hinge shaft and between the lid-side shaft holding sections and the main body-side shaft holding sections and configured to prevent infiltration of liquid from a gap between the lid-side shaft holding sections and the main body-side shaft holding sections into the hinge shaft;
a hinge lid disposed in a hinge shaft insertion port section, into which the hinge shaft is inserted, provided in one of the lid-side shaft holding sections or the main body-side shaft holding sections; and
a second seal member provided in the hinge shaft insertion port section and configured to prevent infiltration of the liquid from a gap between the hinge shaft insertion port section and the hinge lid into the hinge shaft.

2. The endoscope according to claim 1, wherein the first seal member is disposed with an outer surface of the first seal member exposed to an outside.

3. The endoscope according to claim 1, wherein, in a configuration in which one of the main body-side shaft holding sections is provided in a predetermined position of the apparatus main body and a pair of the lid-side shaft holding sections disposed to hold the main body-side shaft holding section is provided in the lid body, the hinge shaft insertion port section is provided in at least one of the two lid-side shaft holding sections.

4. The endoscope according to claim 1, wherein, in a configuration in which one of the lid-side shaft holding sections is provided in a predetermined position of the lid body and a pair of the main body-side shaft holding sections disposed to hold the lid-side shaft holding section is provided in the apparatus main body, the hinge shaft insertion port section is provided in at least one of the two main body-side shaft holding sections.

5. The endoscope according to claim 1, wherein the hinge shaft and the hinge lid are integrated.
